(19) United States
(12) Patent Application Publication
(10) Pub. No.: US 2025/0138011 A1
(43) Pub. Date: May 1, 2025

(54) COMPOSITIONS, KITS, AND METHODS FOR DUPLEX IMMUNOASSAY FOR ANTI-SARS-COV-2 ANTIBODIES

(71) Applicant: Siemens Healthcare Diagnostics Inc., Tarrytown, NY (US)

(72) Inventor: Tie Wei, Wilmington, DE (US)

(73) Assignee: Siemens Healthcare Diagnostics Inc., Tarrytown, NY (US)

(21) Appl. No.: 18/691,351

(22) PCT Filed: Aug. 25, 2022
(86) PCT No.: PCT/US2022/075436

§ 371 Date: Mar. 12, 2024

Related U.S. Application Data
(60) Provisional application No. 63/261,854, filed on Sep. 30, 2021.

(51) Int. Cl. G01N 33/569; B01L 3/00; G01N 21/64; G01N 33/58; G01N 33/68

(52) U.S. Cl. CPC G01N 33/56983; B01L 3/502715; G01N 21/6428; G01N 33/582; G01N 33/6854; B01L 2300/0663; B01L 2300/0864; G01N 2021/6439; G01N 2333/165; G01N 2469/20

(57) ABSTRACT

Kits containing a multiplexed chemiluminescent detection system and microfluidics devices and methods for detecting the presence and/or concentration of anti-SARS-CoV-2 antibodies in a sample are disclosed. The kits, microfluidics devices, and methods utilize singlet oxygen-activatable chemiluminescent compounds in combination with two or more fluorescent molecules that emit light at different wavelengths. In certain non-limiting embodiments, the kits, microfluidics devices, and methods can distinguish between anti-SARS-CoV-2 antibodies generated in response to vaccination from anti-SARS-CoV-2 antibodies generated in response to infection.

### CROSS REFERENCE TO RELATED APPLICATIONS/INCORPORATION BY REFERENCE STATEMENT

Not Applicable.

### STATEMENT REGARDING FEDERALLY SPONSORED RESEARCH OR DEVELOPMENT

Not Applicable.

### BACKGROUND

The field of medical diagnostics utilizes many different forms of assay technologies. When a patient is suspected of being infected with a microorganism (such as, but not limited to, a bacteria or virus), an assay may be performed on a biological sample from the patient to detect antibodies directed to the microorganism that are being produced by the patient's immune system.

When detection of anti-viral or anti-bacterial antigen antibodies (such as, but not limited to, IgG, IgM, and/or IgA) in patient serum and plasma is desired, bridging serology assays have been employed, in which an immobilized viral/bacterial antigen and a labeled viral/bacterial antigen are often used to formulate the assay reagents. In another example, latex particle agglutination assays utilize viral/bacterial antigen-coated latex particles as a single reagent that aggregate in the presence of anti-viral/bacterial antigen antibodies in patient samples.

One example of a commercially used assay is the Luminescent Oxygen Channeling Assay (LOCI®) technology. The LOCI® advanced chemiluminescence assay is described, for example, in U.S. Pat. No. 5,340,716 (Ullman et al.), the entire contents of which are expressly incorporated herein by reference. The currently available LOCI® technology has high sensitivity and uses several reagents. In particular, the LOCI® assay requires that two of these reagents (referred to as a “sensibead” and a “chemibead”) be held by other specific binding partner assay reagents in a manner whereby the sensibead and chemibead are in close proximity to one another to achieve a signal. Upon exposure to light at a certain wavelength, the sensibead releases singlet oxygen, and if the two beads are in close proximity, the singlet oxygen is transferred to the chemibead; this causes a chemical reaction that results in the chemibead giving off light that can be measured at a different wavelength.

In June 2020, the U.S. Food and Drug Administration (FDA) issued an Emergency Use Authorization (EUA) for a laboratory-based total antibody test developed by Siemens Healthineers (Tarrytown, NY) for the detection of the presence of total SARS-CoV-2 antibodies, including IgM and IgG, in blood. This immunoassay is based on the LOCI® platform and is referred to as the CV2T LOCI® immunoassay.

A spike protein on the surface of the SARS-CoV-2 virus enables the virus to penetrate and infect human cells found in multiple organs and blood vessels. The Siemens Healthineers' Total Antibody COV2T CV2T LOCI® immunoassay was designed to detect antibodies to the receptor binding domain (RBD) of spike protein. Some of these antibodies are believed to neutralize the SARS-CoV-2 virus and therefore prevent infection.

In addition, multiple vaccines have been developed and utilized for SARS-CoV-2 that include the spike protein within their focus, including the Pfizer/BioNTech, Moderna, Johnson & Johnson (Janssen), and Oxford-AstraZeneca COVID-19 vaccines that are in wide use. Therefore, immunized patients are now producing antibodies directed to the SARS-CoV-2 spike protein.

Therefore, there is a need in the art for new and improved immunoassays for detecting the presence of SARS-CoV-2 antibodies that can distinguish between antibodies produced in response to vaccination from antibodies produced in response to infection and thereby overcome the disadvantages and defects of the prior art. It is to such assays, as well as kits and microfluidics devices containing same and methods of using same, that the present disclosure is directed.

### BRIEF DESCRIPTION OF THE DRAWINGS

FIG. 1 schematically depicts a SARS-CoV-2 Total (COV2T) assay (Siemens Healthineers, Tarrytown, NY).

FIGS. 2 and 3 schematically depict one non-limiting embodiment of a duplex immunoassay format constructed in accordance with the present disclosure. FIG. 2 depicts the reagents utilized, while FIG. 3 depicts the complexes formed in the presence of anti-SARS-CoV-2 antibodies.

FIGS. 4 and 5 schematically depict another non-limiting embodiment of a duplex immunoassay format constructed in accordance with the present disclosure. FIG. 4 depicts the reagents utilized, while FIG. 5 depicts the complexes formed in the presence of anti-SARS-CoV-2 antibodies.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The terms “analog” and “derivative” are used herein interchangeably and refer to a substance which comprises the same basic carbon skeleton and carbon functionality in its structure as a given compound, but can also contain one or more substitutions thereto. The term “substitution” as used herein will be understood to refer to the replacement of at least one substituent on a compound with a residue R.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “specific binding partner,” as used in particular (but not by way of limitation) herein in the terms “biotin-specific binding partner” or “target analyte-specific binding partner,” will be understood to refer to any molecule capable of specifically associating with biotin or the target analyte, respectively. For example, but not by way of limitation, the binding partner may be an antibody, a receptor, a ligand, aptamers, molecular imprinted polymers (i.e., inorganic matrices), combinations or derivatives thereof, as well as any other molecules capable of specific binding to biotin or the target analyte, respectively.

The term “antibody” is used herein in the broadest sense and refers to, for example, intact monoclonal antibodies and polyclonal antibodies, multi-specific antibodies (e.g., bispecific antibodies), antibody fragments and conjugates thereof that exhibit the desired biological activity of analyte binding (such as, but not limited to, Fab, Fab′, F(ab′)2, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG3, IgG4, IgA1, and IgA2).

The term “hapten” as used herein refers to a small proteinaceous or non-protein antigenic determinant (or “epitope”) which is capable of being recognized by a target analyte-specific binding partner, such as (but not limited to) an antibody. The term “polyhapten” as used herein will be understood to refer to a synthetic molecule that contains multiple epitopes/antigenic determinants attached thereto.

An “analyte” is a macromolecule that is capable of being recognized by an analyte-specific binding partner, such as (but not limited to) an antibody. Both analytes and haptens comprise at least one antigenic determinant or “epitope,” which is the region of the antigen or hapten which binds to the analyte-specific binding partner (i.e., antibody). Typically, the epitope on a hapten is the entire molecule.

Certain non-limiting embodiments of the present disclosure are directed to multiplex assays for the detection anti-SARS-CoV-2 antibodies in samples as well as kits containing same and methods of use thereof. In some assay embodiments, signal producing system (sps) members comprise a sensitizer such as, for example, a photosensitizer, and two or more chemiluminescent-fluorescent molecule compositions (wherein a first chemiluminescent composition generates a signal related to the presence of anti-SARS-CoV-2-spike protein antibodies, whereas at least a second chemiluminescent composition generates a signal related to the presence of anti-SARS-CoV-2 antibodies against another SARS-CoV-2 protein other than the spike protein (such as, but not limited to, the SARS-CoV-2 nucleocapsid protein, envelope protein, or membrane protein)); in these assay embodiments, activation of the sensitizer results in a product that activates the chemiluminescent composition(s), thereby generating a detectable signal that relates to the amount of bound anti-SARS-CoV-2 antibodies being detected. An exemplary (but non-limiting) embodiment of an assay platform on which the present disclosure can be based is the Luminescence Oxygen Channeling Assay (LOCI®; Siemens Healthcare Diagnostics Inc., Tarrytown, NY). The LOCI® assay is described, for example, in U.S. Pat. No. 5,340,716 (Ullman et al.), the entire contents of which are expressly incorporated herein by reference.

Certain non-limiting embodiments of the present disclosure are directed to a kit for performing multiplex assays that utilize a chemiluminescent detection system for determining the presence and/or concentration of multiple anti-SARS-CoV-2 antibodies in a sample. The kit includes: (a) a composition comprising a singlet oxygen-activatable chemiluminescent compound having a first target antigen directly or indirectly bound thereto, and a fluorescent molecule that is excited by the activated chemiluminescent compound; (b) a composition comprising a singlet oxygen-activatable chemiluminescent compound having a second target antigen directly or indirectly bound thereto, and a fluorescent molecule that is excited by the activated chemiluminescent compound, wherein the fluorescent molecule is different from the fluorescent molecule of (a) and emits light at a different wavelength than the fluorescent molecule of (a); (c) a biotinylated first target antigen; (d) a biotinylated second target antigen; and (e) a composition comprising a sensitizer capable of generating singlet oxygen in its excited state and having a biotin-specific binding partner directly or indirectly bound thereto; and wherein the first target antigen is at least a portion of a SARS-CoV-2 spike protein, and wherein the second target antigen is at least a portion of a SARS-CoV-2 protein other than the spike protein.

All or any portion of a SARS-CoV-2 spike protein may be utilized as the first target antigen in accordance with the present disclosure. In certain non-limiting embodiments, the first target antigen includes at least a portion of a SARS-CoV-2 S1 protein, such as (but not limited to) at least a portion of a receptor-binding domain (RBD) of S1 protein.

In one particular (but non-limiting) embodiment, the first target antigen is the receptor binding domain (RBD) of the S1 subunit of SARS-CoV-2 spike protein. The RBD S1 antigen can be obtained from any source known in the art. For example (but not by way of limitation), this particular antigen is commercially available from GenScript (Piscataway, NJ); Meridian Life Sciences, Inc. (Memphis, TN); Sino Biological US Inc. (Wayne, PA); ACRO Biosystems (Newark, DE); Biorbyt, LLC (St. Louis, MO); Icosagen, AS (San Francisco, CA); and Bios Pacific Inc. (Emeryville, CA).

All or any portion of another SARS-CoV-2 protein can be utilized as the second target antigen. In certain non-limiting embodiments, the second target antigen includes at least a portion of a SARS-CoV-2 nucleocapsid protein, at least a portion of a SARS-CoV-2 membrane protein, or at least a portion of a SARS-CoV-2 envelope protein.

A chemiluminescent compound (chemiluminescer) is a compound that is chemically activatable and, as a result of such activation, emits light at a certain wavelength. Examples of chemiluminescers, by way of illustration and not limitation, include: olefins capable of reacting with singlet oxygen or a peroxide to form hydroperoxides or dioxetanes, which can decompose to ketones or carboxylic acid derivatives; stable dioxetanes which can decompose by the action of light; acetylenes which can react with singlet oxygen to form diketones; hydrazones or hydrazides that can form azo compounds or azo carbonyls such as (but not limited to) luminol; and aromatic compounds that can form endoperoxides, for example. As a consequence of the activation reaction, the chemiluminescers directly or indirectly cause the emission of light.

In certain embodiments, the singlet oxygen-activatable chemiluminescent compound may be a substance that undergoes a chemical reaction with singlet oxygen to form a metastabile intermediate species that can decompose with the simultaneous or subsequent emission of light. The composition comprising the chemiluminescent compound may be directly excited by the activated chemiluminescent compound; alternatively, the composition may further comprise at least one fluorescent molecule that is excited by the activated chemiluminescent compound.

A sensitizer is a molecule, usually a compound, that generates a reactive intermediate such as, for example, singlet oxygen, for activation of a chemiluminescent compound. In some non-limiting embodiments, the sensitizer is a photosensitizer. Other sensitizers that can be chemi-activated (by, e.g., enzymes and metal salts) include, by way of example and not limitation, other substances and compositions that can produce singlet oxygen with or without activation by an external light source. For example, certain compounds have been shown to catalyze the conversion of hydrogen peroxide to singlet oxygen and water. Non-limiting examples of other sensitizer substances and compositions include: oxides of the alkaline earth metals Ca, Sr, and Ba; derivatives of elements of groups 3A, 4A, SA, and 6A in d⁰ configuration; oxides of actinides and lanthanides; and oxidizers ClO⁻ BrO⁻, Au³⁺, IO₃⁻, and IO₄⁻; and in particular, molybdate, peroxomolybdate, tungstate, and peroxotungstate ions, and acetonitrile. The following references, which are hereby expressly incorporated by reference in their entirety, provide further disclosure regarding sensitizer substances and compositions that also fall within the scope of the present disclosure: Aubry, J. Am. Chem. Soc., 107:5844-5849 (1985); Aubry, J. Org. Chem., 54:726-728 (1989); Böhme and Brauer, Inorg. Chem., 31:3468-3471 (1992); Niu and Foote, Inorg. Chem., 31:3472-3476 (1992); Nardello et al., Inorg. Chem., 34:4950-4957 (1995); Aubry and Bouttemy, J. Am. Chem. Soc., 119:5286-5294 (1997); and Almeida et al., Anal. Chim. Acta, 482:99-104 (2003); the entire contents of each of which are hereby expressly incorporated herein by reference.

Also included within the scope of photosensitizers are compounds that are not true sensitizers but which on excitation by heat, light, ionizing radiation, or chemical activation will release a molecule of singlet oxygen. Members of this class of compounds include, for example (but not by way of limitation), the endoperoxides such as 1,4-biscarboxyethyl-1,4-naphthalene endoperoxide; 9,10-diphenylanthracene-9,10-endoperoxide; and 5,6,11,12-tetraphenyl naphthalene 5,12-endoperoxide. Heating or direct absorption of light by these compounds releases singlet oxygen.

A photosensitizer is a sensitizer for activation of a photoactive compound, for example, by generation of singlet oxygen by excitation with light. The photosensitizers are photoactivatable and include, e.g., dyes and aromatic compounds, and are usually compounds comprised of covalently bonded atoms, usually with multiple conjugated double or triple bonds. The compounds should absorb light in the wavelength range of from about 200 nm to about 1,100 nm, such as (but not limited to) a range of from about 300 nm to about 1,000 nm or a range of from about 450 nm to 950 nm, with an extinction coefficient at its absorbance maximum greater than 500 M⁻¹ cm⁻¹, or greater than 5,000 M⁻¹ cm⁻¹, or greater than 50,000 M⁻¹ cm⁻¹, at the excitation wavelength. Photosensitizers should be relatively photostable and may not react efficiently with singlet oxygen. Examples of photosensitizers, by way of illustration and not limitation, include: acetone; benzophenone; 9-thioxanthone; eosin; 9,10-dibromoanthracene; methylene blue; metallo-porphyrins such as (but not limited to) hematoporphyrin; phthalocyanines; chlorophylls; rose bengal; and buckminsterfullerene; as well as derivatives of these compounds.

Particular, non-limiting examples of chemiluminescent compounds and photosensitizers that may be utilized in accordance with the present disclosure are set forth in U.S. Pat. No. 5,340,716 (Ullman, et al.), the entire contents of which are hereby expressly incorporated herein by reference.

Any biotin-specific binding partners known in the art or otherwise contemplated herein may be utilized in accordance with the present disclosure. In certain non-limiting embodiments, the biotin-specific binding partner is an antibody against biotin. In other non-limiting embodiments, the biotin-specific binding partner is avidin or an analog thereof.

Any avidin analogs known in the art or otherwise contemplated herein may be utilized in accordance with the present disclosure, so long as the avidin or avidin analog is: (1) capable of association with the sensitizer; (2) capable of binding to the biotinylated analyte-specific binding partner; and (3) capable of binding to biotin that may be present in a sample. Non-limiting examples of avidin analogs that can be utilized in accordance with the present disclosure include those disclosed in Kang et al. (J Drug Target (1995) 3:159-65), the entire contents of which are expressly incorporated herein by reference. Particular non-limiting examples of avidin analogs include avidin, streptavidin, traptavidin, neutral avidin, Neutralite avidin, Neutravidin, Lite-avidin, succinylated avidin, other forms of modified or genetically engineered) avidin, esters, salts, and/or derivatives of any of the above, and the like.

Any fluorescent molecules known in the art that are capable of being excited by the activated chemiluminescent compound and emitting light at a particular, detectable wavelength can be utilized in accordance with the present disclosure as the fluorescent molecules of (a) and (b) (as well as (e), if present), so long as the signals produced by each fluorescent molecule is detectable from the signals produced by the other fluorescent molecules utilized. That is, the fluorescent molecule of (a) must emit light at a wavelength that is sufficiently different from the wavelength at which the fluorescent molecule of (b) emits light so that the two signals can be distinguished from one another when detected simultaneously. In a particular (but non-limiting) example, each fluorescent molecule utilized in accordance with the present disclosure is independently selected from the group consisting of terbium, uranium, samarium, europium, gadolinium, and dysprosium. For example (but not by way of limitation), with respect to the generation of two or three signals that can be distinguished from one another when detected simultaneously, terbium emits light at a wavelength of about 545 nm, uranium emits light at a wavelength of about 612 nm, and samarium emits light at a wavelength of about 645 nm.

Certain non-limiting embodiments of the present disclosure are directed to a kit identical to that described herein above, except that the biotinylated antigens of (c) and (d) are replaced with at least one biotinylated anti-human immunoglobulin (Ig) antibody. Therefore, in this kit, reagents (a) and (b) are identical to the kit above, and then the kit further includes (c) at least one biotinylated anti-human immunoglobulin (Ig) antibody; and (d) a composition comprising a sensitizer and having a biotin-specific binding partner bound thereto (i.e., a composition identical to (e) above). As in the kit described above, the first target antigen is at least a portion of a SARS-CoV-2 spike protein, and the second target antigen is at least a portion of a SARS-CoV-2 protein other than the spike protein.

The anti-human Ig antibodies may specifically bind to any portion of any human immunoglobulin molecules known in the art or otherwise contemplated herein. For example (but not by way of limitation), the antibodies may be directed to human IgG, IgE, IgM, IgD, and/or IgA, and/or any portion thereof (such as, but not limited to, anti-human gamma chain, anti-human H+L, anti-human light chain, and the like). Anti-human Ig antibodies (including, but not limited to, anti-human IgG, anti-human IgM, and/or anti-human IgA antibodies, as well as antibodies that recognize two or more human immunoglobulin antibodies) are well known in the art, are widely commercially available, and have been vastly studied. For example (but not by way of limitation), a few commercial sources of anti-human IgG monoclonal and/or polyclonal antibodies include Rockland Immunochemicals, Inc. (Pottstown, PA); USBiological Life Sciences (Swampscott, MA); Santa Cruz Biotechnology, Inc. (Dallas, TX); Jackson Immuno Research Labs, Inc. (West Grove, PA); Thermo Fisher Scientific (Waltham, MA); and Sigma-Aldrich Corp. (St. Louis, MO). However, this list is not inclusive, and there are many additional commercial sources of anti-human Ig antibodies that can be utilized in accordance with the present disclosure. Thus, a person having ordinary skill in the art will clearly and unambiguously be able to identify and select a variety of anti-human Ig antibodies that can be utilized in accordance with the present disclosure, and as such, no further description of the anti-human Ig antibodies or the characteristics thereof is deemed necessary.

The assay components/reagents of the compositions/kits/microfluidic devices/methods may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in microfluidics devices is described in detail in U.S. Pat. No. 9,244,085 (Samproni), the entire contents of which are hereby expressly incorporated herein by reference.

In addition to the assay components/reagents described in detail herein above, the kits may further contain other reagent(s) for conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the cross-reactivity and stability of the components/reagents. In addition, the kit may include a microfluidics device in which the components/reagents are disposed.

The relative amounts of the various components/reagents in the kits can vary widely to provide for concentrations of the components/reagents that substantially optimize the reactions that need to occur during the assay methods and further to optimize substantially the sensitivity of an assay. Under appropriate circumstances, one or more of the components/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these components. Positive and/or negative controls may also be included with the kit. In addition, the kit can further include a set of written instructions explaining how to use the kit. A kit of this nature can be used in any of the methods described or otherwise contemplated herein.

Certain additional non-limiting embodiments of the present disclosure are directed to a microfluidics device that includes the components of any of the kits described herein above. In particular, certain non-limiting embodiments include a microfluidics device for determining the presence and/or concentrations of multiple anti-SARS-CoV-2 antibodies in a sample. The microfluidics device comprises (i) an inlet channel through which a sample is applied; and (ii) at least a first compartment capable of being in fluidic communication with the inlet channel. The compartment(s) of (ii) contains reagents (a)-(e) of the first kit described herein above (wherein (c) and (d) are biotinylated antigens) or reagents (a)-(d) of the second kit described herein above (wherein (c) is at least one biotinylated anti-human Ig antibody).

Any of the singlet oxygen-activatable chemiluminescent compounds, sensitizers, fluorescent molecules, target antigens, biotin-specific binding partners, and anti-human Ig antibodies described in detail herein above or otherwise contemplated herein may be utilized in the microfluidics devices of the present disclosure.

For example, in certain particular (but non-limiting) embodiments, the singlet oxygen-activatable chemiluminescent compounds of (a) and (b) are substances that undergo a chemical reaction with singlet oxygen to form a metastabile intermediate species that can decompose with the simultaneous or subsequent emission of light.

In particular (but non-limiting) embodiments, the first target antigen is at least a portion of the SARS-CoV-2 S1 protein, such as (but not limited to) the RBD thereof.

In particular (but non-limiting) embodiments, the second target antigen is at least a portion of a SARS-CoV-2 nucleocapsid protein, at least a portion of a SARS-CoV-2 envelope protein, or at least a portion of a SARS-CoV-2 membrane protein.

In particular (but non-limiting) embodiments, the sensitizer is a photosensitizer.

In particular (but non-limiting) embodiments, the biotin-specific binding partner of (iii) is avidin or an analog thereof, or is an antibody against biotin.

In particular (but non-limiting) embodiments, the fluorescent molecules of (a) and (b) are each independently selected from the group consisting of terbium, uranium, samarium, europium, gadolinium, and dysprosium. For example (but not by way of limitation), terbium emits light at a wavelength of about 545 nm, uranium emits light at a wavelength of about 612 nm, and samarium emits light at a wavelength of about 645 nm.

In certain non-limiting embodiments, all of elements (a)-(e) or (a)-(d) (depending on the kit) of (ii) are present in the same compartment. In alternative non-limiting embodiments, elements (a)-(e) or (a)-(d) (depending on the kit) are split between two or more compartments.

The device may be provided with any arrangement of the compartments and distribution of the various components therebetween that allows the device to function in accordance with the present disclosure.

Any of the compartments of the microfluidics device may be sealed to maintain reagent(s) disposed therein in a substantially air tight environment until use thereof; for example, compartments containing lyophilized reagent(s) may be sealed to prevent any unintentional reconstitution of the reagent. The inlet channel and a compartment, as well as two compartments, may be described as being “capable of being in fluidic communication” with one another; this phrase indicates that each of the compartment(s) may still be sealed, but that the two compartments are capable of having fluid flow therebetween upon puncture of a seal formed therein or therebetween.

The microfluidics devices of the present disclosure may be provided with any other desired features known in the art or otherwise contemplated herein. For example, but not by way of limitation, the microfluidics devices of the present disclosure may further include a read chamber; the read chamber may be any of the compartments containing the reagents described herein above, or the read chamber may be in fluidic communication with said compartment. The microfluidics device may further include one or more additional compartments containing other solutions, such as (but not limited to) wash solutions, dilution solutions, excipients, interference solutions, positive controls, negative controls, quality controls, and the like. These additional compartment(s) may be in fluidic communication with one or more of the other compartments. For example, the microfluidics device may further include one or more compartments containing a wash solution, and these compartment(s) may be capable of being in fluidic communication with any other compartment(s) of the device. In another example, the microfluidics device may further include one or more compartments containing an excipient for dissolution of one or more dried reagents, and the compartment(s) may be capable of being in fluidic communication with any other compartment(s) of the device. In yet a further example, the microfluidics device may include one or more compartments containing a dilution solution, and the compartment(s) may be capable of being in fluidic communication with any other compartment(s) of the device.

Certain non-limiting embodiments are also directed to methods for detecting the presence and/or concentration of biotin and at least one additional target analyte in a sample. The methods comprise the following steps.

In the first step, a sample suspected of containing anti-SARS-CoV-2 antibodies is combined, either simultaneously or wholly or partially sequentially, with compositions (a)-(e) of the first kit described herein above or compositions (a)-(d) of the second kit described herein above.

In the second step, the components are incubated together to allow for the binding of (a) and (c) to anti-SARS-CoV-2-spike protein antibodies in the sample (i.e., anti-SARS-CoV-2-RBD antibodies when the first target antigen is the RBD of S1), to allow for the binding of (b) and (d) to anti-SARS-CoV-2 antibodies against the second target antigen in the sample, and to allow for the binding of (c) and (d) to (e). The indirect binding of (a) to (e) results in the formation of a spike protein complex, and the indirect binding of (b) to (e) results in the formation of a second target antigen complex. In each of the spike protein complex and the second target antigen complex, the sensitizer is brought into close proximity to the chemiluminescent compound.

In the third step, the sensitizer is activated to generate singlet oxygen, wherein activation of the sensitizers present in the spike protein complex and in the second target antigen complex causes the activation of the chemiluminescent compound present in each complex.

In the fourth step, the amount of chemiluminescence generated by the activated chemiluminescent compound in the spike protein complex is determined by measuring the amount of light emitted by the fluorescent molecule of (a), wherein the amount of anti-SARS-CoV-2-spike protein antibodies in the sample is proportional to the amount of light emitted.

In the fifth step, the amount of chemiluminescence generated by the activated chemiluminescent compound in the second target antigen complex is determined by measuring the amount of light emitted by the fluorescent molecule of (b), wherein the amount of anti-SARS-CoV-2-second target antigen antibodies in the sample is proportional to the amount of light emitted.

In certain particular (but non-limiting) embodiments, the method may further include a sixth step of determining that anti-SARS-CoV-2 antibodies present in the sample were generated in response to vaccination based on the result of step (4), and determining that anti-SARS-CoV-2 antibodies present in the sample were generated in response to infection based on the result of step (5).

In certain particular (but non-limiting) embodiments, steps (2)-(5) may be repeated one or more times.

Any of the singlet oxygen-activatable chemiluminescent compounds, sensitizers, fluorescent molecules, target antigens, biotin-specific binding partners, and anti-human Ig antibodies described in detail herein above or otherwise contemplated herein may be utilized in the methods of the present disclosure.

For example, in certain particular (but non-limiting) embodiments, the singlet oxygen-activatable chemiluminescent compounds of (a) and (b) are substances that undergo a chemical reaction with singlet oxygen to form a metastabile intermediate species that can decompose with the simultaneous or subsequent emission of light.

In particular (but non-limiting) embodiments, the first target antigen is at least a portion of the SARS-CoV-2 S1 protein, such as (but not limited to) the RBD thereof.

In particular (but non-limiting) embodiments, the second target antigen us at least a portion of a SARS-CoV-2 nucleocapsid protein, at least a portion of a SARS-CoV-2 envelope protein, or at least a portion of a SARS-CoV-2 membrane protein.

In particular (but non-limiting) embodiments, the sensitizer is a photosensitizer, and the activation of the sensitizer in step (3) comprises irradiation with light (such as, but not limited to, irradiation at about 680 nm).

In particular (but non-limiting) embodiments, the fluorescent molecules of (a) and (b) are each independently selected from the group consisting of terbium, uranium, samarium, europium, gadolinium, and dysprosium. For example (but not by way of limitation), terbium emits light at a wavelength of about 545 nm, uranium emits light at a wavelength of about 612 nm, and samarium emits light at a wavelength of about 645 nm.

Any sample for which an assay for the presence and/or concentration of anti-SARS-CoV-2 antibodies is desired can be utilized as the sample in accordance with the methods of the present disclosure. Non-limiting examples of samples include a biological sample such as, but not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof. Particular non-limiting examples include lysed whole blood cells and lysed red blood cells.

As mentioned above, the various components of the method are provided in combination (either simultaneously or sequentially). When the various components of the method are added sequentially, the order of addition of the components may be varied; a person having ordinary skill in the art can determine the particular desired order of addition of the different components to the assay. The simplest order of addition, of course, is to add all the materials simultaneously and determine the signals produced therefrom. Alternatively, each of the components, or groups of components, can be combined sequentially. In certain embodiments, an incubation step may be involved subsequent to one or more additions.

In an alternative (but non-limiting) embodiment, step (1) of the method includes first combining the sample with the biotinylated reagents (i.e., two biotinylated target antigens or at least one biotinylated anti-human Ig antibody) and incubating same before adding the compositions comprising the singlet oxygen-activatable chemiluminescent compounds and the composition comprising the sensitizer. Alternatively, step (1) of the method can include first combining the sample, the biotinylated reagents (i.e., two biotinylated target antigens or at least one biotinylated anti-human Ig antibody), and the compositions comprising the singlet oxygen-activatable chemiluminescent compounds and incubating same before adding the composition comprising the sensitizer.

While particular embodiments of the present disclosure are described as having the LOCI® assay format, it is to be understood that the present disclosure is also directed to other assay formats (and kits, microfluidics devices, and methods of performing same) for which distinction between SARS-CoV-2 antibodies generated in response to vaccination are distinguished from SARS-CoV-2 antibodies generated in response to infection is desired. For example (but not by way of limitation), the present disclosure also includes assay formats where different signal molecules such as (but not limited to) different antibodies linked to different enzymes that generate signals at different wavelengths can be utilized in place of the chemiluminescent compound-containing compositions described herein above.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1

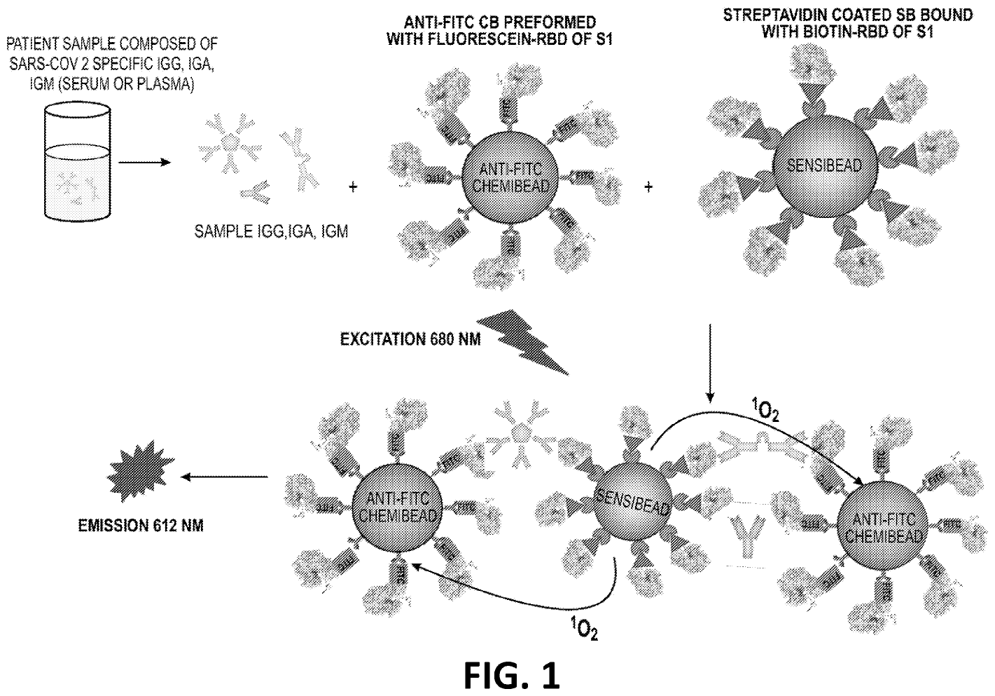

In June 2020, the U.S. Food and Drug Administration (FDA) issued an Emergency Use Authorization (EUA) for a laboratory-based total antibody test developed by Siemens Healthineers (Malvern, PA) for the detection of the presence of SARS-CoV-2 antibodies, including IgM, IgA, and IgG, in blood. A spike protein on the surface of the SARS-CoV-2 virus enables the virus to penetrate and infect human cells found in multiple organs and blood vessels. The Siemens Healthineers' Total Antibody COV2T assay is shown in FIG. 1 and was designed to detect antibodies to the RBD of spike protein. Some of these antibodies are believed to neutralize the SARS-CoV-2 virus and therefore prevent infection. Multiple potential vaccines in development for SARS-CoV-2 include the spike protein within their focus.

The assay format of FIG. 1 utilizes two reagents that both contain SARS-CoV-2 antigens: a first reagent comprising anti-FITC chemibeads preformed with the fluorescein-labeled receptor binding domain of the S1 subunit of SARS-CoV-2 spike protein (RBD of S1), and a second reagent that is formed by the binding of universal streptavidin-coated sensibeads with biotinylated-RBD of S1.

The immunoassay format of the present disclosure adds (i) a second chemibead having a second antigen from a different SARS-CoV-2 protein attached thereto and a different fluorescent molecule associated therewith, and (ii) a biotinylated second antigen for interaction with the streptavidin-coated sensibeads. In this manner, two LOCI® immunoassays occur in a single reaction with a sample.

In this Example, the use of a duplex immunoassay to simultaneously detect anti-nucleocapsid (NC) and anti-spike protein antibodies from SARS-CoV-2 in a patient's sample is described. A software algorithm has also been developed to report if the anti-SARS-CoV-2 antibodies present in a patient's sample were generated due to infection and/or vaccination, as well as a titer for each antibody.

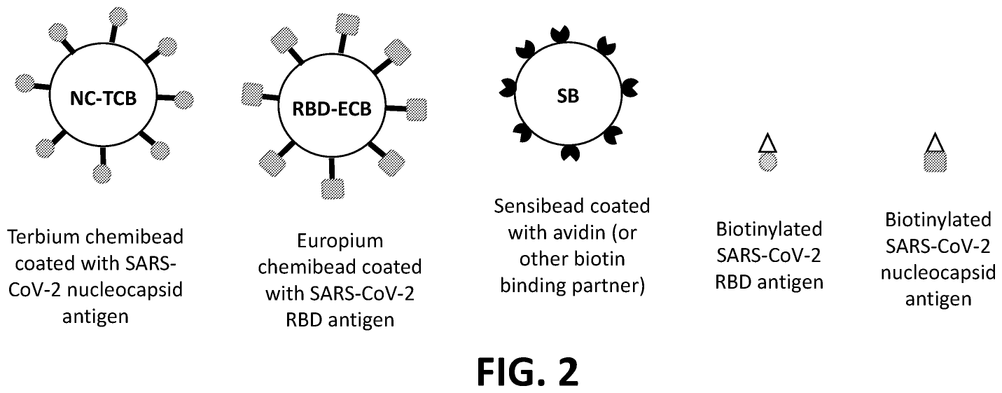

In this Example, and as shown in FIG. 2, a LOCI assay was constructed with the following reagents:

First, universal sensibeads that are coated with streptavidin in the usual manner.

Second, two types of biotinylated SARS-CoV-2 viral antigens are produced: a biotinylated nucleocapsid protein or a portion thereof (biotin-NC); and a biotinylated spike protein or a portion thereof (could be S1 or RBD thereof=biotin-RBD will be used herein to represent this component).

Third, two types of Chemibeads (CB) are produced: CB dyed with Terbium (TCB) and coated with nucleocapsid protein or a portion thereof (NC-TCB); and CB dyed with Europium (ECB) and coated with spike protein or a portion thereof (RBD-ECB). Note that these particular combinations of dye and antigen are solely for purposes of example only. Any chemibead dye can be utilized with any antigen protein. The only requirement is that the two chemibeads present have different dyes that fluoresce at different wavelengths.

The chemibeads with antigen attached may be formed in the same manner as the chemibead utilized in the commercially available Total-SARS-CoV-2 assay mentioned above (i.e., by conjugating fluorescein-labeled-RBD of S1 to anti-FITC chemibeads). Alternatively, the antigen may be directly conjugated to the surface of the chemibeads.

The fluorescent molecules of the two types of chemibeads (i.e., Terbium and Europium, or other fluorescent molecules) are different from one another and emit light at different wavelengths; in this manner, assays for antibodies to both SARS-CoV-2 antigens can be performed simultaneously in the same reaction vessel, as complexes containing antibodies to the first target antigen are detected at a different wavelength from complexes containing antibodies to the second target antigen. Alternatively, the two antibody assays can be performed sequentially but in the same reaction vessel (i.e., by passing the single reaction vessel to two different detectors for signal measurements at the different wavelengths).

In one non-limiting embodiment, the following reaction sequence is used. However, it will be understood that the order of addition of reagents can easily be changed and optimized by a person having ordinary skill in the art. Therefore, the following reaction sequence is solely for purposes of example and is non-limiting of the methods of the present disclosure.

The biotinylated first and second target antigens (i.e., biotin-NC and biotin-RBD) are combined with the patient sample suspected of containing anti-SARS-CoV-2 antibodies (anti-SC2 Abs). Then the two chemibeads (i.e., NC-TCB and RBD-ECB) are added, and the reaction mixture is incubated to allow for sandwich formation. When anti-SARS-CoV-2 antibodies against the nucleocapsid protein (aNC-Abs) are present, the following sandwich is formed:
Biotin-NC:aNC-Ab:NC-TCB.
   When anti-SARS-CoV-2 antibodies against the RBD of S1 (aRBD-Abs) are present, the following sandwich is formed:
Biotin-RBD:aRBD-Ab:RBD-ECB.

Then the sensibeads (SB) are added in excess, and the reaction mixture is incubated so that the sandwiches formed above are captured by sensibeads. When anti-SARS-CoV-2 antibodies against the nucleocapsid protein (aNC-Abs) are present, the following complex is formed:
SB:biotin-NC:aNC-Ab:NC-TCB.
   When anti-SARS-CoV-2 antibodies against the RBD of S1 (aRBD-Abs) are present, the following complex is formed:
SB:biotin-RBD:aRBD-Ab:RBD-ECB.

Note that sensibeads are universal reagents that have multiple biotin-specific binding partners bound thereto. Therefore, while the two sandwich complexes immediately above are described herein above as being two separate, discrete sandwich complexes, it is theoretically possible that two types of sandwiches (i.e., biotinylated target antigen:anti-SARS-CoV-2 antibody:target analyte-coated chemibead) could be bound to the same sensibead.

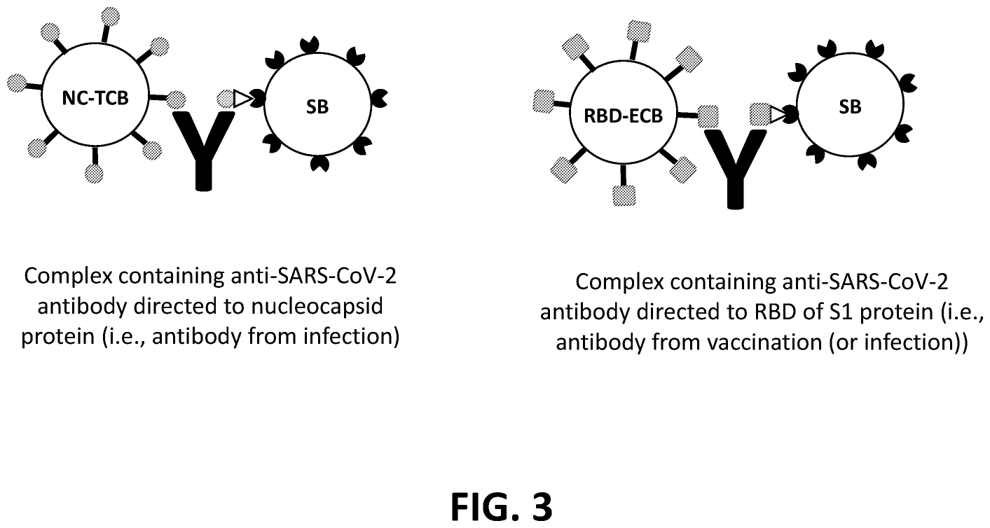

Once the reaction mixture is incubated for sufficient time to allow for formation of the above two complexes in the presence of the target antibodies (as shown in FIG. 3), the chemiluminescence reaction is trigged by excitation light at 680 nm to release singlet oxygen (¹O₂). In the reaction mixture, one or both of (i) and (ii) could occur:
(i) when SB:biotin-NC:aNC-Ab:NC-TCB complexes are present, due to spatial closeness, singlet oxygen diffuses from the sensibead to the chemibead and triggers photon release at 545 nm, and this can be detected by PMT tailored to receive light at 545 nm (this can be as having a spinning filter); and
(ii) when SB:biotin-RBD:aRBD-Ab:RBD-ECB complexes are present, due to spatial closeness, singlet oxygen diffuses from the sensibead to the chemibead and triggers photon release at 612 nm, and this can be detected by PMT tailored to receive light at 612 nm (this is the current LOCI PMT detection).

Typically, two basic scenarios will be detected: a signal from (ii) alone or a combination of signals from (i) and (ii). If the signal generated by the complexes of (i) are detected, either alone or in combination with (ii), then the antibodies are due to infection. If only the signal generated by the complexes of (ii) are present, then the antibodies are due to vaccination.

In one non-limiting embodiment, the signals generated by the two types of complexes of (i) and (ii) are then fed into the following algorithm programmed in the instrument's software:
(a) if only 612 nm signal is received, the assay detected only anti-SARS-CoV-2 RBD antibodies, and the antibodies are due to vaccination.
(b) if only 545 nm signal is received, the assay detected only anti-SARS-CoV-2 NC antibodies, and the antibodies are due to infection, most likely to be from early stage of infection.
(c) if both 612 nm and 545 nm signals are detected, then at least some of the antibodies are due to infection. For example (but not by way of limitation), the following scenarios may be present:
   1) if the 612 nm signal is stronger than the 545 nm signal, the patient could have antibodies generated from infection alone or from both vaccination and infection.
   2) if the 612 nm signal is not as strong as or weaker than the 545 nm signal, the patient has an infection.

Under certain non-limiting conditions, it may be desirable to output information distinguishing between (b) and (c) as above. Under other non-limiting conditions, it may be desirable to only report a “no infection” versus “infection” status for the sample that only distinguishes the signal of (a) from the combination of (b)/(c), respectively; that is, the detection of any anti-SARS-CoV-2 NC antibodies in the sample should output the reporting of an “infection” status for the sample.

While the use of biotinylated target antigens (i.e., biotin-NC and biotin-RBD) are described herein above, it will be understood that these two biotinylated reagents can be replaced with one or more biotinylated anti-human Ig antibodies as an alternative in the duplex immunoassay format.

The RBD portion of SARS-CoV-2 S protein has been utilized in the above Example, as neutralizing antibodies from patients bind to the RBD to prevent RBD from binding to ACE2 (a receptor) on human cell membranes; therefore, immunoassays that utilize RBD as the viral antigen are detecting patient antibodies against RBD of spike protein. Both the Pfizer-BioNTech and Moderna COVID-19 vaccines that have been authorized in the US under emergency use authorizations (EUAs) in 2021 are made of entire S protein. As such, the scope of the present disclosure includes the use of the entire S protein as the antigen in any of the duplex immunoassays described herein. However, because of the homology of the entire S protein to other viruses, a more specific signal may be seen with using CoV-2 RBD as the antigen rather than the entire S protein.

While chemibeads containing Terbium and Europium are described herein above, it will be understood that combinations of fluorescent molecules that fluoresce at wavelengths that differ over a wider range may be used. For example, a chemibead dyed with Samarium dye (SCB), which releases photons at 645 nm, may be used in lieu of the ECB, and the PMT tailored accordingly.

Further, the multiplex assays of the present disclosure can be adapted to simultaneously detect antibodies to three or more SARS-CoV-2 target analytes. For each additional antibody to target analyte to be detected, additional components like the chemibead antigen and biotinylated antigen are added, wherein the fluorescent molecule present in the additional component like differs from the first and second fluorescent molecules in that it emits light at a different and separately detectable wavelength than the first and second fluorescent molecules. In this manner, antibodies to two, three, four, five, six, seven, eight, nine, ten, or more SARS-CoV-2 target antigens can be detected in a single reaction, so long as the chemibeads used to detect each contain different fluorescent molecules that each emit light at different and separately detectable wavelengths; as such, the limiting factor in how many different antibodies can be detected in a single reaction are the number of fluorescent molecules available that function as described herein.

The duplex immunoassay format of the present disclosure is useful in determining whether a patient's anti-SARS-CoV-2 antibodies are from infection or vaccination. This assay format is also useful in determining whether vaccination is effective not only in people without infection but also in people with past COVID-19 infection.

### Example 2

The duplex immunoassay of this Example is similar to the assay format described above in Example 1. However, this immunoassay format differs in that, instead of utilizing biotinylated target antigens, at least one biotinylated anti-human immunoglobulin antibody is utilized instead to bind to the anti-SARS-CoV-2 antibodies in the sample to form a sandwich with the respective chemibead and then capture the formed sandwiches by the sensibeads.

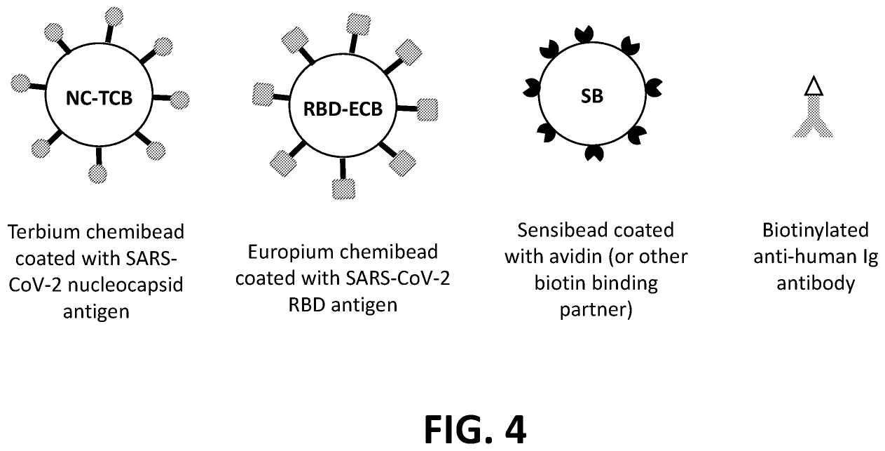

In this Example, and as shown in FIG. 4, a LOCI assay is constructed with the following reagents:

First, universal sensibeads are obtained as described in Example 1.

Second, two types of chemibeads are produced (NC-TCB and RBD-ECB) as described in Example 1.

Third, at least one biotinylated anti-human Ig antibody is produced.

In one non-limiting embodiment, the following reaction sequence is used. However, it will be understood that the order of addition of reagents can easily be changed and optimized by a person having ordinary skill in the art. Therefore, the following reaction sequence is solely for purposes of example and is non-limiting of the methods of the present disclosure.

The at least one biotinylated anti-human Ig antibody (Anti-hIg Ab) is combined with the patient sample suspected of containing anti-SARS-CoV-2 antibodies (anti-SC2 Abs). Then the two chemibeads (i.e., NC-TCB and RBD-ECB) are added, and the reaction mixture is incubated to allow for sandwich formation. When anti-SARS-CoV-2 antibodies against the nucleocapsid protein (aNC-Abs) are present, the following sandwich is formed:
Biotin-Anti-hIg Ab:aNC-Ab:NC-TCB.
   When anti-SARS-CoV-2 antibodies against the RBD of S1 (aRBD-Abs) are present, the following sandwich is formed:
Biotin-Anti-hIg Ab:aRBD-Ab:RBD-ECB.

Then the sensibeads (SB) are added in excess, and the reaction mixture is incubated so that the sandwiches formed above are captured by sensibeads. When anti-SARS-CoV-2 antibodies against the nucleocapsid protein (aNC-Abs) are present, the following complex is formed:
SB:biotin-Anti-hIg Ab:aNC-Ab:NC-TCB.
   When anti-SARS-CoV-2 antibodies against the RBD of S1 (aRBD-Abs) are present, the following complex is formed:
SB:biotin-anti-hIg Ab:aRBD-Ab:RBD-ECB.

Note that sensibeads are universal reagents that have multiple biotin-specific binding partners bound thereto. Therefore, while the two sandwich complexes immediately above are described herein above as being two separate, discrete sandwich complexes, it is theoretically possible that two types of sandwiches (i.e., biotinylated anti-human Ig antibody:anti-SARS-CoV-2 antibody:target analyte-coated chemibead) could be bound to the same sensibead.

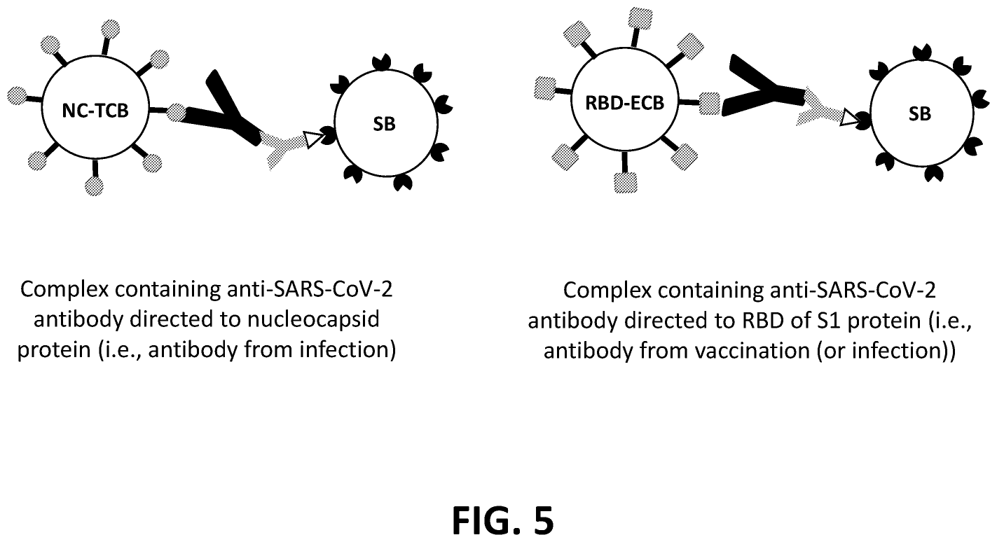

Once the reaction mixture is incubated for sufficient time to allow for formation of the above two complexes in the presence of the target antibodies (as shown in FIG. 5), the chemiluminescence reaction is trigged by excitation light at 680 nm to release singlet oxygen (¹O₂). In the reaction mixture, one or both of (i) and (ii) could occur:
(i) when SB:biotin-anti-hIg Ab:aNC-Ab:NC-TCB complexes are present, due to spatial closeness, singlet oxygen diffuses from the sensibead to the chemibead and triggers photon release at 545 nm, and this can be detected by PMT tailored to receive light at 545 nm (this can be as having a spinning filter); and
(ii) when SB:biotin-anti-hIg Ab:aRBD-Ab:RBD-ECB complexes are present, due to spatial closeness, singlet oxygen diffuses from the sensibead to the chemibead and triggers photon release at 612 nm, and this can be detected by PMT tailored to receive light at 612 nm (this is the current LOCI PMT detection).

The signals generated by the two types of complexes of (i) and (ii) are then fed into the following algorithm programmed in the instrument's software:
(a) if only 612 nm signal is received, the assay detected only anti-SARS-CoV-2 RBD antibodies, and the antibodies are due to vaccination.
(b) if only 545 nm signal is received, the assay detected only anti-SARS-CoV-2 NC antibodies, and the antibodies are due to infection, most likely to be from early stage of infection.
(c) if both 612 nm and 545 nm signals are detected, then the following scenarios:
   1) if the 612 nm signal is stronger than the 545 nm signal, the patient has antibodies generated from both vaccination and infection.
   2) if the 612 nm signal is not as strong as or weaker than the 545 nm signal, the patient has an infection.

While chemibeads containing Terbium and Europium are described herein above, it will be understood that combinations of fluorescent molecules that fluoresce at wavelengths that differ over a wider range may be used. For example, a chemibead dyed with Samarium dye (SCB), which releases photons at 645 nm, may be used in lieu of the ECB, and the PMT tailored accordingly.

Thus, in accordance with the present disclosure, there have been provided compositions, kits, and devices, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A kit for performing a multiplex assay that utilizes a chemiluminescent detection system for determining the presence and/or concentrations of multiple anti-SARS-CoV-2 antibodies in a sample, the kit comprising:
(a) a composition comprising:
a singlet oxygen-activatable chemiluminescent compound having a first target antigen directly or indirectly bound thereto; and
a fluorescent molecule that is excited by the activated chemiluminescent compound;
(b) a composition comprising:
a singlet oxygen-activatable chemiluminescent compound having a second target antigen directly or indirectly bound thereto; and
a fluorescent molecule that is excited by the activated chemiluminescent compound, wherein the fluorescent molecule is different from the fluorescent molecule of (a) and emits light at a different wavelength than the fluorescent molecule of (a);
(c) a biotinylated first target antigen;
(d) a biotinylated second target antigen; and
(e) a composition comprising a sensitizer capable of generating singlet oxygen in its excited state and having a biotin-specific binding partner directly or indirectly bound thereto; and
wherein the first target antigen is at least a portion of a SARS-CoV-2 spike protein, and wherein the second target antigen is at least a portion of a SARS-CoV-2 protein other than the spike protein.

2. The kit of claim 1, wherein the first target antigen comprises a receptor-binding domain (RBD) of S1 protein.

3. The kit of claim 1, wherein the second target antigen is at least a portion of a SARS-CoV-2 nucleocapsid protein.

4. The kit of claim 1, wherein the second target antigen is at least a portion of a SARS-CoV-2 membrane protein or at least a portion of a SARS-CoV-2 envelope protein.

5. The kit of claim 1, wherein at least one of:
the singlet oxygen-activatable chemiluminescent compound of each of (a) and (b) is a substance that undergoes a chemical reaction with singlet oxygen to form a metastabile intermediate species that can decompose with the simultaneous or subsequent emission of light;
the sensitizer is a photosensitizer; and
the biotin-specific binding partner of (e) is selected from the group consisting of avidin, an analog of avidin, and an antibody against biotin.

6. The kit of claim 1, wherein the fluorescent molecules of (a) and (b) are each independently selected from the group consisting of terbium, uranium, samarium, europium, gadolinium, and dysprosium.

7. A microfluidics device for determining the presence and/or concentrations of multiple anti-SARS-CoV-2 antibodies in a sample, the microfluidics device comprising:
(i) an inlet channel through which a sample is applied;
(ii) at least a first compartment capable of being in fluidic communication with the inlet channel and containing:
(a) a composition comprising:
a singlet oxygen-activatable chemiluminescent compound having a first target antigen directly or indirectly bound thereto; and
a fluorescent molecule that is excited by the activated chemiluminescent compound;
(b) a composition comprising:
a singlet oxygen-activatable chemiluminescent compound having a second target antigen directly or indirectly bound thereto; and
a fluorescent molecule that is excited by the activated chemiluminescent compound, wherein the fluorescent molecule is different from the fluorescent molecule of (a) and emits light at a different wavelength than the fluorescent molecule of (a);
(c) a biotinylated first target antigen;
(d) a biotinylated second target antigen; and
(e) a composition comprising a sensitizer capable of generating singlet oxygen in its excited state and having a biotin-specific binding partner directly or indirectly bound thereto; and
wherein the first target antigen is at least a portion of a SARS-CoV-2 spike protein, and wherein the second target antigen is at least a portion of a SARS-CoV-2 protein other than the spike protein.

8. The microfluidics device of claim 7, wherein the first target antigen comprises a receptor-binding domain (RBD) of S1 protein.

9. The microfluidics device of claim 7, wherein the second target antigen is at least a portion of a SARS-CoV-2 nucleocapsid protein.

10. The microfluidics device of claim 7, wherein the second target antigen is at least a portion of a SARS-CoV-2 membrane protein or at least a portion of a SARS-CoV-2 envelope protein.

11. The microfluidics device of claim 7, wherein (a)-(e) are present in the same compartment.

12. The microfluidics device of claim 7, wherein (a)-(e) are split between two or more compartments.

13. The microfluidics device of claim 7, wherein at least one of:
the singlet oxygen-activatable chemiluminescent compound of each of (a) and (b) is a substance that undergoes a chemical reaction with singlet oxygen to form a metastabile intermediate species that can decompose with the simultaneous or subsequent emission of light;
the sensitizer is a photosensitizer; and
the biotin-specific binding partner of (e) is selected from the group consisting of avidin, an analog of avidin, and an antibody against biotin.

14. The microfluidics device of claim 7, wherein the fluorescent molecules of (a) and (b) are each independently selected from the group consisting of terbium, uranium, samarium, europium, gadolinium, and dysprosium.

15. A method for detecting the presence and/or concentrations of multiple anti-SARS-CoV-2 antibodies in a sample, the method comprising the steps of:
(1) combining, either simultaneously or wholly or partially sequentially, a sample suspected of SARS-CoV-2 antibodies with:
(a) a composition comprising:
a singlet oxygen-activatable chemiluminescent compound having a first target antigen directly or indirectly bound thereto; and
a fluorescent molecule that is excited by the activated chemiluminescent compound;
(b) a composition comprising:
a singlet oxygen-activatable chemiluminescent compound having a second target antigen directly or indirectly bound thereto; and
a fluorescent molecule that is excited by the activated chemiluminescent compound, wherein the fluorescent molecule is different from the fluorescent molecule of (a) and emits light at a different wavelength than the fluorescent molecule of (a);
(c) a biotinylated first target antigen;
(d) a biotinylated second target antigen; and
(e) a composition comprising a sensitizer capable of generating singlet oxygen in its excited state and having a biotin-specific binding partner directly or indirectly bound thereto; and
wherein the first target antigen is at least a portion of a SARS-CoV-2 spike protein, and wherein the second target antigen is at least a portion of a SARS-CoV-2 protein other than the spike protein;
(2) allowing the binding of (a) and (c) to anti-SARS-CoV-2-spike protein antibodies in the sample, allowing the binding of (b) and (d) to anti-SARS-CoV-2 antibodies against the second target antigen in the sample, and allowing the binding of (c) and (d) to (e), wherein the indirect binding of (a) to (e) results in the formation of a spike protein complex, and wherein the indirect binding of (b) to (e) results in the formation of a second target antigen complex, and wherein in each of the spike protein complex and the second target antigen complex the sensitizer is brought into close proximity to the chemiluminescent compound; and
(3) activating the sensitizer to generate singlet oxygen, wherein activation of the sensitizers present in the spike protein complex and in the second target antigen complex causes the activation of the chemiluminescent compound present in each complex;
(4) determining the amount of chemiluminescence generated by the activated chemiluminescent compound in the spike protein complex by measuring the amount of light emitted by the fluorescent molecule of (a), wherein the amount of anti-SARS-CoV-2-spike protein antibodies in the sample is proportional to the amount of light emitted; and
(5) determining the amount of chemiluminescence generated by the activated chemiluminescent compound in the second target antigen complex by measuring the amount of light emitted by the fluorescent molecule of (b), wherein the amount of anti-SARS-CoV-2-second target antigen antibodies in the sample is proportional to the amount of light emitted.

16. The method of claim 15, further comprising the step of:
(6) determining that anti-SARS-CoV-2 antibodies present in the sample were generated in response to vaccination based on the result of step (4), and determining that anti-SARS-CoV-2 antibodies present in the sample were generated in response to infection based on the result of step (5).

17. The method of claim 15, further comprising repeating steps (2)-(5).

18. The method of claim 15, wherein the first target antigen comprises a receptor-binding domain (RBD) of S1 protein.

19. The method of claim 15, wherein the second target antigen is at least a portion of a SARS-CoV-2 nucleocapsid protein.

20. The method of claim 15, wherein the second target antigen is at least a portion of a SARS-CoV-2 membrane protein or at least a portion of a SARS-CoV-2 envelope protein.

21. The method of claim 15, wherein at least one of:
the singlet oxygen-activatable chemiluminescent compound of each of (a) and (b) is a substance that undergoes a chemical reaction with singlet oxygen to form a metastabile intermediate species that can decompose with the simultaneous or subsequent emission of light;
the sensitizer of (e) is a photosensitizer, and wherein the activation of the sensitizer in step (3) comprises irradiation with light; and
the biotin-specific binding partner of (e) is selected from the group consisting of avidin, an analog of avidin, and an antibody against biotin.

22. The method of claim 15, wherein the sample is a biological sample selected from the group consisting of whole blood or any portion thereof, urine, saliva, sputum, cerebrospinal fluid, skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof.

23. A kit for performing a multiplex assay that utilizes a chemiluminescent detection system for determining the presence and/or concentrations of multiple anti-SARS-CoV-2 antibodies in a sample, the kit comprising:
(a) a composition comprising:
a singlet oxygen-activatable chemiluminescent compound having a first target antigen directly or indirectly bound thereto; and
a fluorescent molecule that is excited by the activated chemiluminescent compound;
(b) a composition comprising:
a singlet oxygen-activatable chemiluminescent compound having a second target antigen directly or indirectly bound thereto; and
a fluorescent molecule that is excited by the activated chemiluminescent compound, wherein the fluorescent molecule is different from the fluorescent molecule of (a) and emits light at a different wavelength than the fluorescent molecule of (a);
(c) at least one biotinylated anti-human immunoglobulin (Ig) antibody; and
(d) a composition comprising a sensitizer capable of generating singlet oxygen in its excited state and having a biotin-specific binding partner directly or indirectly bound thereto; and
wherein the first target antigen is at least a portion of a SARS-CoV-2 spike protein, and wherein the second target antigen is at least a portion of a SARS-CoV-2 protein other than the spike protein.

24. The kit of claim 23, wherein the first target antigen comprises a receptor-binding domain (RBD) of S1 protein.

25. The kit of claim 23, wherein the second target antigen is at least a portion of a SARS-CoV-2 nucleocapsid protein.

26. The kit of claim 23, wherein the second target antigen is at least a portion of a SARS-CoV-2 membrane protein or at least a portion of a SARS-CoV-2 envelope protein.

27. The kit of claim 23, wherein at least one of:
the singlet oxygen-activatable chemiluminescent compound of each of (a) and (b) is a substance that undergoes a chemical reaction with singlet oxygen to form a metastabile intermediate species that can decompose with the simultaneous or subsequent emission of light;
the sensitizer is a photosensitizer; and
the biotin-specific binding partner of (d) is selected from the group consisting of avidin, an analog of avidin, and an antibody against biotin.

28. The kit of claim 23, wherein the fluorescent molecules of (a) and (b) are each independently selected from the group consisting of terbium, uranium, samarium, europium, gadolinium, and dysprosium.

29. A microfluidics device for determining the presence and/or concentrations of multiple anti-SARS-CoV-2 antibodies in a sample, the microfluidics device comprising:
(i) an inlet channel through which a sample is applied;
(ii) at least a first compartment capable of being in fluidic communication with the inlet channel and containing:
(a) a composition comprising:
a singlet oxygen-activatable chemiluminescent compound having a first target antigen directly or indirectly bound thereto; and
a fluorescent molecule that is excited by the activated chemiluminescent compound;
(b) a composition comprising:
a singlet oxygen-activatable chemiluminescent compound having a second target antigen directly or indirectly bound thereto; and
a fluorescent molecule that is excited by the activated chemiluminescent compound, wherein the fluorescent molecule is different from the fluorescent molecule of (a) and emits light at a different wavelength than the fluorescent molecule of (a);
(c) at least one biotinylated anti-human immunoglobulin (Ig) antibody; and
(d) a composition comprising a sensitizer capable of generating singlet oxygen in its excited state and having a biotin-specific binding partner directly or indirectly bound thereto; and
wherein the first target antigen is at least a portion of a SARS-CoV-2 spike protein, and wherein the second target antigen is at least a portion of a SARS-CoV-2 protein other than the spike protein.

30. The microfluidics device of claim 29, wherein the first target antigen comprises a receptor-binding domain (RBD) of S1 protein.

31. The microfluidics device of claim 29, wherein the second target antigen is at least a portion of a SARS-CoV-2 nucleocapsid protein.

32. The microfluidics device of claim 29, wherein the second target antigen is at least a portion of a SARS-CoV-2 membrane protein or at least a portion of a SARS-CoV-2 envelope protein.

33. The microfluidics device of claim 29, wherein (a)-(d) are present in the same compartment.

34. The microfluidics device of claim 29, wherein (a)-(d) are split between two or more compartments.

35. The microfluidics device of claim 29, wherein at least one of:
the singlet oxygen-activatable chemiluminescent compound of each of (a) and (b) is a substance that undergoes a chemical reaction with singlet oxygen to form a metastabile intermediate species that can decompose with the simultaneous or subsequent emission of light;
the sensitizer is a photosensitizer; and
the biotin-specific binding partner of (d) is selected from the group consisting of avidin, an analog of avidin, and an antibody against biotin.

36. The microfluidics device of claim 29, wherein the fluorescent molecules of (a) and (b) are each independently selected from the group consisting of terbium, uranium, samarium, europium, gadolinium, and dysprosium.

37. A method for detecting the presence and/or concentration of multiple anti-SARS-CoV-2 antibodies in a sample, the method comprising the steps of:
(1) combining, either simultaneously or wholly or partially sequentially, a sample suspected of SARS-CoV-2 antibodies with:
(a) a composition comprising:
a singlet oxygen-activatable chemiluminescent compound having a first target antigen directly or indirectly bound thereto; and
a fluorescent molecule that is excited by the activated chemiluminescent compound;
(b) a composition comprising:
a singlet oxygen-activatable chemiluminescent compound having a second target antigen directly or indirectly bound thereto; and
a fluorescent molecule that is excited by the activated chemiluminescent compound, wherein the fluorescent molecule is different from the fluorescent molecule of (a) and emits light at a different wavelength than the fluorescent molecule of (a);
(c) at least one biotinylated anti-human immunoglobulin (Ig) antibody; and
(d) a composition comprising a sensitizer capable of generating singlet oxygen in its excited state and having a biotin-specific binding partner directly or indirectly bound thereto; and
wherein the first target antigen is at least a portion of a SARS-CoV-2 spike protein, and wherein the second target antigen is at least a portion of a SARS-CoV-2 protein other than the spike protein;
(2) allowing the binding of (a) and (c) to anti-SARS-CoV-2-spike protein antibodies in the sample, allowing the binding of (b) and (c) to anti-SARS-CoV-2 antibodies against the second target antigen in the sample, and allowing the binding of (c) to (d), wherein the indirect binding of (a) to (d) results in the formation of a spike protein complex, and wherein the indirect binding of (b) to (d) results in the formation of a second target antigen complex, and wherein in each of the spike protein complex and the second target antigen complex the sensitizer is brought into close proximity to the chemiluminescent compound; and
(3) activating the sensitizer to generate singlet oxygen, wherein activation of the sensitizers present in the spike protein complex and in the second target antigen complex causes the activation of the chemiluminescent compound present in each complex;
(4) determining the amount of chemiluminescence generated by the activated chemiluminescent compound in the spike protein complex by measuring the amount of light emitted by the fluorescent molecule of (a), wherein the amount of anti-SARS-CoV-2-spike protein antibodies in the sample is proportional to the amount of light emitted; and
(5) determining the amount of chemiluminescence generated by the activated chemiluminescent compound in the second target antigen complex by measuring the amount of light emitted by the fluorescent molecule of (b), wherein the amount of anti-SARS-CoV-2-second target antigen antibodies in the sample is proportional to the amount of light emitted.

38. The method of claim 37, further comprising the step of:
(6) determining that anti-SARS-CoV-2 antibodies present in the sample were generated in response to vaccination based on the result of step (4), and determining that anti-SARS-CoV-2 antibodies present in the sample were generated in response to infection based on the result of step (5).

39. The method of claim 37, further comprising repeating steps (2)-(5).

40. The method of claim 37, wherein the first target antigen comprises a receptor-binding domain (RBD) of S1 protein.

41. The method of claim 37, wherein the second target antigen is at least a portion of a SARS-CoV-2 nucleocapsid protein.

42. The method of claim 37, wherein the second target antigen is at least a portion of a SARS-CoV-2 membrane protein or at least a portion of a SARS-CoV-2 envelope protein.

43. The method of claim 37, wherein at least one of:
the singlet oxygen-activatable chemiluminescent compound of each of (a) and (b) is a substance that undergoes a chemical reaction with singlet oxygen to form a metastabile intermediate species that can decompose with the simultaneous or subsequent emission of light;
the sensitizer of (d) is a photosensitizer, and wherein the activation of the sensitizer in step (3) comprises irradiation with light; and
the biotin-specific binding partner of (d) is selected from the group consisting of avidin, an analog of avidin, and an antibody against biotin.

44. The method of claim 37, wherein the sample is a biological sample selected from the group consisting of whole blood or any portion thereof, urine, saliva, sputum, cerebrospinal fluid, skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof.
